(19) United States
(12) Patent Application Publication
(10) Pub. No.: US 2025/0064722 A1
(43) Pub. Date: Feb. 27, 2025

(54) ANTI-BCMA CAR T CELL COMPOSITIONS

(71) Applicant: 2seventy bio, Inc., Cambridge, MA (US)

(72) Inventors: Travis Quigley, Cohasset, MA (US); Robert Ross, Brookline, MA (US)

(21) Appl. No.: 18/820,674

(22) Filed: Aug. 30, 2024

Related U.S. Application Data
(62) Division of application No. 16/346,393, filed on Apr. 30, 2019, now Pat. No. 12,109,234.
(60) Provisional application No. 62/514,401, filed on Jun. 2, 2017.
(60) Provisional application No. 62/417,840, filed on Nov. 4, 2016.

(51) Int. Cl. A61K 9/00; A61K 35/17; A61K 39/00; A61P 35/00; C07K 14/705; C07K 14/725; C07K 16/28

(52) U.S. Cl. CPC A61K 9/0019; A61K 35/17; A61K 39/4611; A61K 39/4631; A61K 39/464417; A61P 35/00; C07K 14/7051; C07K 14/70517; C07K 14/70578; C07K 16/2878; A61K 2239/38; A61K 2239/48; C07K 2317/622; C07K 2319/03

(57) ABSTRACT

The invention provides improved anti-BCMA CAR T cell compositions for adoptive T cell therapy for relapsed/refractory multiple myeloma.

### CROSS REFERENCE TO RELATED APPLICATIONS

This application is a U.S. national phase application of International PCT Patent Application No. PCT/US2017/059989, filed on Nov. 3, 2017, which claims the benefit under 35 U.S.C. § 119(e) of U.S. Provisional Application No. 62/514,401, filed Jun. 2, 2017, and U.S. Provisional Application No. 62/417,840, filed Nov. 4, 2016, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_079_02WO_ST25.txt. The text file is 27 KB, was created on Nov. 3, 2017, and is being submitted electronically via EFS-Web, concurrent with the filing of the specification.

### BACKGROUND

### Technical Field

The present invention relates to improved compositions and methods for treating B cell related conditions. More particularly, the invention relates to improved compositions comprising therapeutic doses of anti-BCMA chimeric antigen receptor (CAR) T cells to treat relapsed/refractory multiple myeloma.

### Description of the Related Art

Chimeric Antigen Receptor (CAR) T-cells are molecules that combine antibody-based specificity for a desired antigen with a T cell receptor-activating intracellular domain to generate a chimeric protein that exhibits a specific anti-cancer immune activity. CARs endow a patient's own T cells with the ability to recognize and kill cancer cells directly. CAR T cell technology has developed rapidly, but has yet to realize its therapeutic potential. Several obstacles remain to the clinical application of CAR T cells to hematological malignancies.

Most CAR T cell therapies applied to hematological malignancies are associated with cytokine release syndrome (CRS) or cytokine storm. CRS is specific for adoptive cellular immunotherapies, manifests with septic shock-like symptoms, and may ultimately result in patient death. In fact, a number of clinical trials were temporarily suspended by the FDA due to CRS-related deaths in CAR T-cell trials. CRS can be rapid, unpredictable and currently, there is no consensus on prevention and management of CRS. However, one characteristic that contributes to CRS is the therapeutic cell dose of CAR T cells.

CAR T cells are “living drugs” that have the ability to multiply in vivo—after infusion and home to bone marrow. For example, in a seminal case study from UPenn, published in 2011, as low as 1.5×10⁵ CAR T cells, given to chronic lymphocytic leukemia (CLL) patients, expanded more than 1000 times in vivo over time. Thus, CAR T cell therapy presents a unique set of challenges with respect to dosing. There is no uniform consensus on CAR T cell infusion dosage and influence factors, including tumor load, efficacy and side effects. Small dose infusions may not obtain the ideal curative effect, but may instead induce tumor antigen deletion, and antigen escape. Large dose infusions and serious tumor load will increase CRS and tumor lysis syndrome. Because it is a living drug, determining the correct therapeutic dose is unpredictable. Conventional drug-body interaction concepts of pharmacodynamics and pharmacokinetics can't readily be applied to CAR T cell therapy because CAR T cells are a dynamic, living, and persistent drug.

Some challenges to dose selection include, but are not limited to, the paucity of correlative animal models, first in human products have limited “a priori” information, in-vivo expansion of humans cell is unpredictable, the limitations in “borrowing” safety data from first generation CAR T products, extrapolating safety data from related products (TILs, “similar” TCR redirected cells, “similar” class of CAR T product, and extrapolating safety data using the same product in histologically different tumor type(s).

Accordingly, current approaches to CAR T cell dosing do not appear to be sufficiently mature to predict therapeutically effective CAR T cell dose among T cells harboring different CARs for use in the same indication and among the same CAR T cells for use in different indications.

### BRIEF SUMMARY

The invention generally provides improved anti-BCMA CAR T cell compositions and methods of using the same to treat relapsed/refractory multiple myeloma.

In various embodiments, a composition is contemplated comprising a therapeutically effective amount of anti-B cell maturation antigen (BCMA) chimeric antigen receptor (CAR) T cells, wherein the therapeutically effective amount is greater than about 5.0×10⁷ anti-BCMA CAR T cells, and the anti-BCMA CAR comprises the amino acid sequence set forth in SEQ ID NO: 9.

In particular embodiments, the composition further comprises a pharmaceutically acceptable carrier.

In certain embodiments, the composition is formulated in a solution comprising 50:50 PlasmaLyte A to CryoStor CS10.

In various embodiments, the therapeutically effect amount is at least about 15.0×10⁷ anti-BCMA CAR T cells.

In particular embodiments, the therapeutically effect amount is at least about 45.0×10⁷ anti-BCMA CAR T cells.

In some embodiments, the therapeutically effect amount is at least about 80.0×10⁷ anti-BCMA CAR T cells.

In certain embodiments, the therapeutically effect amount is at least about 12.0×10 anti-BCMA CAR T cells.

In particular embodiments, the therapeutically effect amount is between about 5.0×10⁷ anti-BCMA CAR T cells and about 15.0×10⁷ anti-BCMA CAR T cells.

In various embodiments, the therapeutically effect amount is between about 5.0×10⁷ anti-BCMA CAR T cells and about 45.0×10⁷ anti-BCMA CAR T cells.

In some embodiments, the therapeutically effect amount is between about 5.0×10⁷ anti-BCMA CAR T cells and about 80.0×10⁷ anti-BCMA CAR T cells.

In particular embodiments, the therapeutically effect amount is between about 5.0×10⁷ anti-BCMA CAR T cells and about 12.0×10⁸ anti-BCMA CAR T cells.

In certain embodiments, the therapeutically effect amount is between about 15.0×10⁷ anti-BCMA CAR T cells and about 45.0×10⁷ anti-BCMA CAR T cells.

In some embodiments, the therapeutically effect amount is between about 15.0×10⁷ anti-BCMA CAR T cells and about 80.0×10⁷ anti-BCMA CAR T cells.

In various embodiments, the therapeutically effect amount is between about 15.0×10⁷ anti-BCMA CAR T cells and about 12.0×10⁸ anti-BCMA CAR T cells.

In particular embodiments, the anti-BCMA CAR T cells are transduced with a lentiviral vector encoding the anti-BCMA CAR.

In certain embodiments, the lentiviral vector copy number (VCN) is about 2.0 copies per anti-BCMA CAR T cell.

In certain embodiments, the lentiviral vector is a human immunodeficiency virus 1 (HIV-1) vector.

In particular embodiments, a pharmaceutical composition is contemplated comprising a population of T cells transduced with a lentivirus encoding an anti-BCMA CAR, wherein population of T cells comprises greater than about 5.0×10⁷ anti-BCMA CAR T cells, and the anti-BCMA CAR comprises the amino acid sequence set forth in SEQ ID NO: 9.

In some embodiments, the composition further comprising a therapeutically acceptable carrier.

In various embodiments, the composition comprises at least about 15.0×10⁷ anti-BCMA CAR T cells.

In particular embodiments, the composition comprises at least about 45.0×10⁷ anti-BCMA CAR T cells.

In some embodiments, the composition comprises at least about 80.0×10⁷ anti-BCMA CAR T cells.

In certain embodiments, the composition comprises at least about 12.0×10 anti-BCMA CAR T cells.

In various embodiments, the composition comprises about 15.0×10⁷ anti-BCMA CAR T cells.

In particular embodiments, the composition comprises about 45.0×10⁷ anti-BCMA CAR T cells.

In some embodiments, the composition comprises about 80.0×10⁷ anti-BCMA CAR T cells.

In certain embodiments, the composition comprises about 12.0×10 anti-BCMA CAR T cells.

In particular embodiments, the composition comprises between about 5.0×10⁷ anti-BCMA CAR T cells and about 15.0×10⁷ anti-BCMA CAR T cells.

In various embodiments, the composition comprises between about 5.0×10⁷ anti-BCMA CAR T cells and about 45.0×10⁷ anti-BCMA CAR T cells.

In particular embodiments, the composition comprises between about 5.0×10⁷ anti-BCMA CAR T cells and about 80.0×10⁷ anti-BCMA CAR T cells.

In some embodiments, the composition comprises between about 5.0×10⁷ anti-BCMA CAR T cells and about 12.0×10⁸ anti-BCMA CAR T cells.

In various embodiments, the composition comprises between about 15.0×10⁷ anti-BCMA CAR T cells and about 45.0×10⁷ anti-BCMA CAR T cells.

In certain embodiments, the composition comprises between about 15.0×10⁷ anti-BCMA CAR T cells and about 80.0×10⁷ anti-BCMA CAR T cells.

In some embodiments, the composition comprises between about 15.0×10⁷ anti-BCMA CAR T cells and about 12.0×10⁸ anti-BCMA CAR T cells.

In particular embodiments, the T cells comprise CD8+ T cells.

In various embodiments, a method of treating a subject that has been diagnosed with relapsed/refractory multiple myeloma is provided comprising administering the subject a composition contemplated herein.

In particular embodiments, a method of treating a subject that has relapsed/refractory multiple myeloma is provided, comprising administering the subject the composition contemplated herein.

In certain embodiments, the composition is administered in a single dose.

In certain embodiments, the composition is intravenously administered.

In some embodiments, the multiple myeloma was refractory to at least three treatment regimens, including a proteasome inhibitor and an immunomodulatory agent, prior to the administration of the composition.

In particular embodiments, the multiple myeloma was double-refractory to one or more treatment regimens, prior to the administration of the composition.

In some embodiments, the subject was treated with daratumumab, lenalidomide, pomalidomide, bortezomib, and/or carfilzomib, prior to the administration of the composition.

In certain embodiments, the subject received an autologous hematopoietic stem cell transplant, prior to the administration of the composition.

In particular embodiments, the subject was lymphodepleted with cyclophosphamide 300 mg/m² and fludarabine 30 mg/m².

### BRIEF DESCRIPTION OF SEVERAL VIEWS OF THE DRAWINGS

FIG. 1 shows a schematic of a murine B cell maturation antigen (muBCMA) CAR construct.

FIG. 2A shows comparable levels of expansion for anti-BCMA CAR T cells manufactured from PBMCs of 11 individual donors compared to a matched culture of untransduced donor T cells.

FIG. 2B shows comparable levels of lentiviral transduction efficiency (VCN) in anti-BCMA CAR T cells manufactured from PBMCs of 11 individual donors.

FIG. 2C shows comparable anti-BCMA CAR expression by flow cytometry in anti-BCMA CAR T cells manufactured from PBMCs of 11 individual donors.

FIG. 2D shows comparable levels of IFNγ release when anti-BCMA CAR T cells manufactured from PBMCs of 11 individual donors were exposed to BCMA-expressing K562 cells.

FIG. 3 shows clinical response over time of relapsed refractory multiple myeloma patients treated with anti-BCMA CAR T cells.

### BRIEF DESCRIPTION OF THE SEQUENCE IDENTIFIERS

SEQ ID NOs: 1-3 set forth amino acid sequences of exemplary light chain CDR sequences for BCMA CARs contemplated herein.

SEQ ID NOs: 4-6 set forth amino acid sequences of exemplary heavy chain CDR sequences for BCMA CARs contemplated herein.

SEQ ID NO: 7 sets forth an amino acid sequence of an exemplary light chain sequences for BCMA CARs contemplated herein.

SEQ ID NO: 8 sets forth an amino acid sequence of an exemplary heavy chain sequences for BCMA CARs contemplated herein.

SEQ ID NO: 9 sets forth an amino acid sequence of an exemplary BCMA CAR contemplated herein.

SEQ ID NO: 10 set forth a polynucleotide sequence that encode an exemplary BCMA CAR contemplated herein.

SEQ ID NO: 11 sets forth the amino acid sequence of human BCMA.

SEQ ID NO: 12-22 set for the amino acid sequence of various linkers.

SEQ ID NOs: 23-35 set for the amino acid sequence of protease cleavage sites and self-cleaving polypeptide cleavage sites.

SEQ ID NO: 36 sets for the polynucleotide sequence of a vector encoding a BCMA CAR.

### DETAILED DESCRIPTION

### A. Overview

Significant challenges exist in the art to predicting a therapeutically effective CAR T cell dose with respect to a particular CAR and its target indication. The present disclosure generally relates to improved anti-BCMA CAR T cell compositions and methods for treating relapsed/refractory multiple myeloma.

B cell maturation antigen (BCMA, also known as CD269 or tumor necrosis factor receptor superfamily, member 17; TNFRSF17 is a member of the tumor necrosis factor receptor superfamily (see, e.g., Thompson et al., J. Exp. Medicine, 192(1): 129-135, 2000, and Mackay et al., Annu. Rev. Immunol, 21: 231-264, 2003. BCMA binds B-cell activating factor (BAFF) and a proliferation inducing ligand (APRIL) (see, e.g., Mackay et al., 2003 and Kalled et al., Immunological Reviews, 204: 43-54, 2005). Among nonmalignant cells, BCMA has been reported to be expressed mostly in plasma cells and subsets of mature B-cells (see, e.g., Laabi et al., EMBO J., 77(1): 3897-3904, 1992; Laabi et al., Nucleic Acids Res., 22(7): 1147-1154, 1994; Kalled et al., 2005; O'Connor et al., J. Exp. Medicine, 199(1): 91-97, 2004; and Ng et al., J. Immunol., 73(2): 807-817, 2004. Mice deficient in BCMA are healthy and have normal numbers of B cells, but the survival of long-lived plasma cells is impaired (see, e.g., O'Connor et al., 2004; Xu et al., Mol. Cell. Biol, 21(12): 4067-4074, 2001; and Schiemann et al., Science, 293(5537): 2 111-21 14, 2001). BCMA RNA has been detected universally in multiple myeloma cells and in other lymphomas, and BCMA protein has been detected on the surface of plasma cells from multiple myeloma patients by several investigators (see, e.g., Novak et al., Blood, 103(2): 689-694, 2004; Neri et al., Clinical Cancer Research, 73(19): 5903-5909, 2007; Bellucci et al., Blood, 105(10): 3945-3950, 2005; and Moreaux et al., Blood, 703(8): 3148-3157, 2004.

In various embodiments, the improved adoptive cell therapy compositions contemplated herein comprise therapeutically effective doses of anti-BCMA CAR T cells that unexpectedly expand and elicit clinical responses in vivo, in the absence of severe cytokine release syndrome CRS.

In particular embodiments, the improved compositions comprising therapeutically effective doses of anti-BCMA CAR T cells contemplated herein are used to treat relapsed/refractory multiple myeloma.

The practice of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 20%, 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±20%, ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

“Polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full-length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides contemplated herein comprise a signal (or leader) sequence at the N-terminal end of the protein, which co-translationally or post-translationally directs transfer of the protein. Illustrative examples of suitable signal sequences useful in CARs disclosed herein include, but are not limited to the IgG1 heavy chain signal sequence and the CD8α signal sequence. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acid of a CAR as disclosed herein. In particular embodiments, the term “polypeptide” further includes variants, fragments, and fusion polypeptides

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the CARs by introducing one or more substitutions, deletions, additions and/or insertions into a binding domain, hinge, TM domain, co-stimulatory signaling domain or primary signaling domain of a CAR polypeptide. Preferably, polypeptides of the invention include polypeptides having at least about 65%, 70%, 75%, 85%, 90%, 95%, 98%, or 99% amino acid identity thereto.

Polypeptides include “polypeptide fragments.” Polypeptide fragments refer to a polypeptide, which can be monomeric or multimeric and that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long.

Fusion polypeptides and fusion proteins refer to a polypeptide having at least two, three, four, five, six, seven, eight, nine, or ten or more polypeptide segments.

As used herein, the terms “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides of the invention include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present invention contemplates, in part, polynucleotides comprising expression vectors, viral vectors, and transfer plasmids, and compositions, and cells comprising the same.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

The term “vector” is used herein to refer to a nucleic acid molecule capable transferring or transporting another nucleic acid molecule.

The term “lentiviral vector” refers to a viral vector or plasmid containing structural and functional genetic elements, or portions thereof, including LTRs that are primarily derived from a lentivirus.

In particular embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. Self-inactivation is preferably achieved through in the introduction of a deletion in the U3 region of the 3′ LTR of the vector DNA, i.e., the DNA used to produce the vector RNA. Thus, during reverse transcription, this deletion is transferred to the 5′ LTR of the proviral DNA. In particular embodiments, it is desirable to eliminate enough of the U3 sequence to greatly diminish or abolish altogether the transcriptional activity of the LTR, thereby greatly diminishing or abolishing the production of full-length vector RNA in transduced cells. In the case of HIV based lentivectors, it has been discovered that such vectors tolerate significant U3 deletions, including the removal of the LTR TATA box (e.g., deletions from −418 to −18), without significant reductions in vector titers.

### C. Anti-BCMA Chimeric Antigen Receptors

In various embodiments, genetically engineered receptors that redirect cytotoxicity of T cells toward BCMA expressing cells are provided. These genetically engineered receptors referred to herein as anti-BCMA chimeric antigen receptors (CARs).

Anti-BCMA CARs contemplated in particular embodiments, comprise a signal peptide, an anti-BCMA scFv, a CD8α hinge domain, a CD8α transmembrane domain, a 4-1BB co-stimulatory domain, and a CD3ζ primary signaling domain.

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding.

“V_{H}” or “VH” refer to the variable region of an immunoglobulin heavy chain. “V_{L}” or “VL” refer to the variable region of an immunoglobulin light chain. In various embodiments, the scFv comprises a light chain sequence set forth in SEQ ID NO: 7 and a heavy chain sequence set forth in SEQ ID NO: 8.

Light and heavy chain variable regions contain three hypervariable regions, also called “complementarity-determining regions” or “CDRs.” In particular embodiments, the scFv light chain comprises the CDRL1-3 sequences set forth in SEQ ID NOs: 1-3, and the scFv heavy chain comprises the CDRH1-3 sequences set forth in SEQ ID NOs: 4-6.

In certain embodiments, the CARs contemplated herein may comprise linker residues between the various domains, e.g., added for appropriate spacing and conformation of the molecule. In particular embodiments, the linker comprises the following amino acid sequence: GSTSGSGKPGSGEGSTKG (SEQ ID NO: 22) (Cooper et al., Blood, 101(4): 1637-1644 (2003)).

In particular embodiments, the hinge domain comprises a CD8α hinge region.

The “transmembrane domain” is the portion of the CAR that fuses the extracellular binding portion and intracellular signaling domain and anchors the CAR to the plasma membrane of the T cell. In particular embodiments, the transmembrane domain comprises a CD8α transmembrane region.

In particular embodiments, a CAR contemplated herein comprises a “co-stimulatory signaling domain” and a “primary signaling domain.” As used herein, the term, “co-stimulatory signaling domain,” or “co-stimulatory domain,” refers to an intracellular signaling domain of a co-stimulatory molecule. Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs. In particular embodiments, the anti-BCMA CAR comprises a CD137 co-stimulatory signaling domain and a CD3ζ primary signaling domain.

In preferred embodiments, the anti-BCMA CAR comprises the amino acid sequence set forth in SEQ ID NO: 9.

In particular embodiments, a polynucleotide encoding an anti-BCMA CAR polypeptides set forth in SEQ ID NO: 9 is provided.

The polynucleotides contemplated in particular embodiments, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

In particular embodiments, a vector encoding an anti-BCMA CAR contemplated herein is a viral vector, will include exogenous, endogenous, or heterologous control sequences such as promoters and/or enhancers.

In a particular embodiment, the vector comprises a myeloproliferative sarcoma virus enhancer, negative control region deleted, d1587rev primer-binding site substituted (MND) promoter (Challita et al., J Virol. 69(2):748-55 (1995)) operably linked to a polynucleotide encoding an anti-BCMA CAR contemplated herein.

In particular embodiments, a cell (e.g., a T cell) is transduced with a retroviral vector, e.g., a lentiviral vector, encoding an anti-BCMA CAR contemplated herein. In preferred embodiments, the lentiviral vector is HIV (human immunodeficiency virus; including HIV type 1). In particular preferred embodiments, the lentiviral vectors is a SIN HIV-1 vector.

### D. Anti-BCMA Car T Cells

The present disclosure contemplates, in particular embodiments, T cells comprising an anti-BCMA CAR, e.g., anti-BCMA CAR T cells. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4⁻CD8⁻ T cell, or any other subset of T cells. Other illustrative populations of T cells suitable for use in particular embodiments include naïve T cells and memory T cells.

T cells of the invention can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells of the invention are allogeneic.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

T cells can be genetically modified following isolation using known methods, or the T cells can be activated and expanded (or differentiated in the case of progenitors) in vitro prior to being genetically modified. In a particular embodiment, the T cells are genetically modified with the chimeric antigen receptors contemplated herein (e.g., transduced with a viral vector comprising a nucleic acid encoding a CAR) and then are activated and expanded in vitro. In various embodiments, T cells can be activated and expanded before or after genetic modification to express a CAR.

In one embodiment, T cells expressing an anti-BCMA CAR are cryopreserved such that the cells remain viable upon thawing. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). The preferred cooling rate is 1° to 3° C./minute. After at least two hours, the T cells have reached a temperature of −80° C. and can be placed directly into liquid nitrogen (−196° C.) for permanent storage such as in a long-term cryogenic storage vessel.

### E. Compositions and Formulations

The compositions contemplated herein comprise a therapeutically effective amount of anti-BCMA CAR T cells. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount, and more preferably a therapeutically effective amount, of anti-BCMA CAR-expressing T cells contemplated herein.

As used herein, the term “amount” or “dose” refers to “an amount effective,” “a dose effective,” “an effective amount,” or “an effective dose” of an anti-BCMA CAR T cell sufficient to achieve a beneficial or desired prophylactic or therapeutic result, including clinical results.

A “therapeutically effective amount” or “therapeutically effective dose” of an anti-BCMA CAR T cell is also one in which any toxic or detrimental effects of an anti-BCMA CAR T cell, e.g., CRS, are outweighed by the therapeutically beneficial effects. The term “therapeutically effective amount” includes an amount that is effective to “treat” a subject (e.g., a patient). In one embodiment, the therapeutically effective dose is the minimal effective dose (MED) of anti-BCMA CAR T cells to treat multiple myeloma in a subject. In one embodiment, the therapeutically effective dose is the maximum tolerated dose (MTD) of anti-BCMA CAR T cells that does not lead to unresolvable CRS in a subject.

In particular embodiments, compositions are preferably formulated for parenteral administration, e.g., intravascular (intravenous or intraarterial), administration. In a preferred embodiment, the compositions contemplated herein are intravenously infused into the subject in a single dose.

In one embodiment, the amount of anti-BCMA CAR+ T cells in a composition administered to a subject is at least about 5.0×10⁷ cells, at least about 15.0×10⁷ cells, at least about 45.0×10⁷ cells, at least about 80.0×10⁷ cells, or at least about 12.0×10⁸ cells.

In one embodiment, the amount of anti-BCMA CAR+ T cells in a composition administered to a subject is greater than about 5.0×10⁷ cells, greater than about 15.0×10⁷ cells, greater than about 45.0×10⁷ cells, greater than about 80.0×10⁷ cells, or greater than about 12.0×10⁸ cells.

In one embodiment, the amount of anti-BCMA CAR+ T cells in a composition administered to a subject is between about 5.0×10⁷ cells to about 15.0×10⁷ cells, between about 5.0×10⁷ cells to about 45.0×10⁷ cells, between about 5.0×10⁷ cells to about 80.0×10⁷ cells, or between about 5.0×10⁷ cells to about 12.0×10⁸ cells.

In one embodiment, the amount of anti-BCMA CAR+ T cells in a composition administered to a subject is between about 15.0×10⁷ cells to about 45.0×10⁷ cells, between about 15.0×10⁷ cells to about 80.0×10⁷ cells, or between about 15.0×10⁷ cells to about 12.0×10⁸ cells.

In one embodiment, the amount of anti-BCMA CAR+ T cells in a composition administered to a subject is at least 5.0×10⁷ cells ±20%, at least 15.0×10⁷ cells ±20%, at least 45.0×10⁷ cells ±20%, at least 80.0×10⁷ cells, ±20% or at least 12.0×10⁸ cells ±20%.

In one embodiment, the amount of anti-BCMA CAR+ T cells in a composition administered to a subject is greater than 5.0×10⁷ cells ±20%, at least 15.0×10⁷ cells ±20%, at least 45.0×10⁷ cells ±20%, at least 80.0×10⁷ cells ±20%, or at least 12.0×10⁸ cells ±20%.

In one embodiment, the amount of anti-BCMA CAR+ T cells in a composition administered to a subject is between 5.0×10⁷ cells ±20% to 15.0×10⁷ cells ±20%, between 5.0×10⁷ cells ±20% to 45.0×10⁷ cells ±20%, between 5.0×10⁷ cells ±20% to 80.0×10⁷ cells ±20%, or between 5.0×10⁷ cells ±20% to 12.0×10⁸ cells ±20%.

In one embodiment, the amount of anti-BCMA CAR+ T cells in a composition administered to a subject is between 15.0×10⁷ cells ±20% to 45.0×10⁷ cells ±20%, between 15.0×10⁷ cells ±20% to 80.0×10⁷ cells ±20%, or between 15.0×10⁷ cells ±20% to 12.0×10⁸ cells ±20%.

For uses provided herein, the cells are generally in a volume of a liter or less, can be 500 mLs or less, even 250 mLs or 100 mLs or less.

In particular embodiments, pharmaceutical compositions comprise a therapeutically effective amount of anti-BCMA CAR T cells, in combination with one or more pharmaceutically or physiologically acceptable carriers, diluents or excipients.

Pharmaceutical compositions comprising a therapeutically effective dose of anti-BCMA CAR T cells may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, anti-BCMA CAR T cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising anti-BCMA CAR T cells contemplated herein are formulated in a solution comprising PlasmaLyte A.

In another preferred embodiment, compositions comprising anti-BCMA CAR T cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising anti-BCMA CAR T cells contemplated herein are formulated in a solution comprising 50:50 PlasmaLyte A to CryoStor CS10.

### F. Therapeutic Methods

Methods for treating multiple myeloma contemplated herein comprise administering a composition comprising a therapeutically effective amount of T cells that express an anti-BCMA CAR to a subject.

As used herein, the terms “individual” and “subject” are often used interchangeably and refer to a human that has multiple myeloma. In preferred embodiments, a subject refers to a human that has relapsed/refractory multiple myeloma.

As used herein, the term “patient” refers to a subject that has been diagnosed with multiple myeloma and more preferably, relapsed/refractory multiple myeloma.

As used herein “treatment” or “treating,” includes any beneficial or desirable effect on the symptoms or pathology of multiple myeloma and more preferably, relapsed/refractory multiple myeloma, and may include even minimal reductions in one or more measurable markers of multiple myeloma and more preferably, relapsed/refractory multiple myeloma.

Treatment can involve optionally either the reduction or amelioration of symptoms of a multiple myeloma, or the delaying of the progression of a multiple myeloma. “Treatment” does not necessarily indicate complete eradication or cure of a multiple myeloma, or associated symptoms thereof.

As used herein, “prevent,” and similar words such as “prevented,” “preventing” etc., indicate an approach for preventing, inhibiting, or reducing the likelihood of relapse of a multiple myeloma.

Multiple myeloma is a B cell malignancy of mature plasma cell morphology characterized by the neoplastic transformation of a single clone of these types of cells. These plasma cells proliferate in BM and may invade adjacent bone and sometimes the blood.

Variant forms of multiple myeloma include overt multiple myeloma, smoldering multiple myeloma, plasma cell leukemia, non-secretory myeloma, IgD myeloma, osteosclerotic myeloma, solitary plasmacytoma of bone, and extramedullary plasmacytoma (see, for example, Braunwald, et al. (eds), Harrison's Principles of Internal Medicine, 15th Edition (McGraw-Hill 2001)).

In particular embodiments, compositions comprising a therapeutically effective amount of anti-BCMA CAR T cells are administered to a subject to treat relapsed/refractory multiple myeloma. “Relapse” refers to the diagnosis of return, or signs and symptoms of return, of a cancer after a period of improvement or remission. “Refractory” refers to a cancer that is resistant to, or non-responsive to, therapy with a particular therapeutic agent. A cancer can be refractory from the onset of treatment (i.e., non-responsive to initial exposure to the therapeutic agent), or as a result of developing resistance to the therapeutic agent, either over the course of a first treatment period or during a subsequent treatment period.

In particular embodiments, compositions contemplated herein are administered to a subject with relapsed/refractory multiple myeloma that has been unsuccessfully treated with one, two, three or more treatments, including at least one proteasome inhibitor and/or an immunomodulatory drug (IMiD). In one embodiment, the subject's multiple myeloma is refractory to three treatment regimens, including at least one proteasome inhibitor and an IMiD. In one embodiment, the subject's multiple myeloma is double-refractory to one or more treatment regimens.

Illustrative examples of proteasome inhibitors to which subject's multiple myeloma is refractory include, but are not limited to, bortezomib, and carfilzomib.

Illustrative examples of IMiDs to which subject's multiple myeloma is refractory include, but are not limited to thalidomide, lenalidomide, and pomalidomide.

Illustrative examples of other treatments, to which multiple myeloma may be refractory include, but are not limited to, dexamethasone, and antibody-based therapies selected from the group consisting of elotuzumab, daratumumab, MOR03087, isatuximab, bevacizumab, cetuximab, siltuximab, tocilizumab, elsilimomab, azintrel, rituximab, tositumomab, milatuzumab, lucatumumab, dacetuzumab, figitumumab, dalotuzumab, AVE1642, tabalumab, pembrolizumab, pidilizumab, and nivolumab.

In one embodiment, the subject's multiple myeloma is refractory to treatment with daratumumab.

In particular embodiments, the subject's multiple myeloma is refractory to treatment with an IMiD, a proteasome inhibitor, and dexamethasone.

Methods contemplated herein, may further comprise treating a subject with relapsed/refractory multiple myeloma with an autologous hematopoietic stem cell transplant, prior to the administration of the anti-BCMA CAR T cell composition.

Methods contemplated herein, may further comprise lymphodepleting the subject prior to administration of an anti-BCMA CAR T cell composition contemplated herein, e.g., for example, the lymphodepleting chemotherapy ends 1-4 days (e.g., 1, 2, 3, or 4 days) prior to the administration. In particular embodiments, the lymphodepletion comprises administering one or more of melphalan, cytoxan, cyclophosphamide, and fludarabine. In one embodiment the subject is lymphodepleted with cyclophosphamide 300 mg/m² and fludarabine 30 mg/m² prior to administration of an anti-BCMA CAR T cell composition contemplated herein.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Construction of BCMA Cars

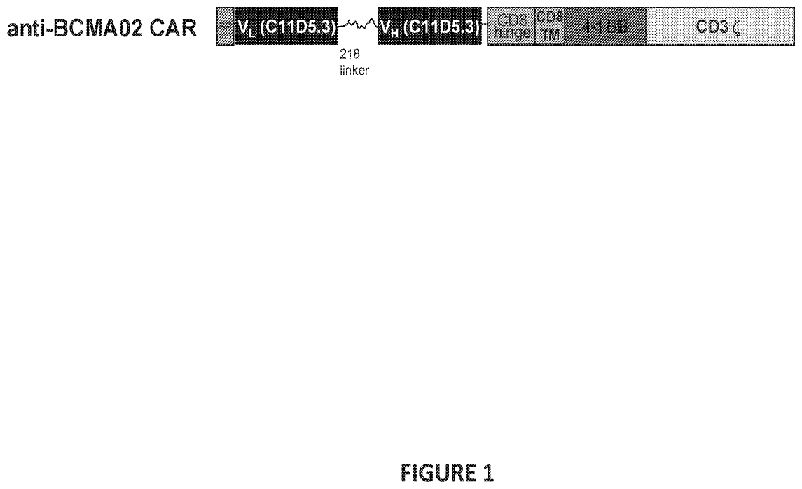

CARs containing murine ann-BCMA scFv antibodies were designed to contain an MND promoter operably linked to ano-BMCA scFv, a hinge and transmembrane domain from CD8α and a CD137 co-stimulatory domains followed by the intracellular signaling domain of the CD3ζ chain. See, e.g., FIG. 1. The anti-BMCA CAR shown in FIG. 1 comprises a CD8α signal peptide (SP) sequence for the surface expression on T cells. The polynucleotide sequence of an exemplary anti-BMCA CAR is set forth in SEQ ID NO: 10; an exemplary polypeptide sequences of a ant-BMCA CAR is set forth in SEQ ID NO: 9; and a vector map of an exemplary CAR construct is shown in FIG. 1. Table 3 shows the Identity, Genbank Reference, Source Name and Citation for the various nucleotide segments of an anti-BMCA CAR lentiviral vector.

TABLE 3

| Nucleotides | Identity | GenBank Reference | Source Name | Citation |
|---|---|---|---|---|
| 1-185 | pUC19 plasmid | Accession #L09137.2 | pUC19 | New England |
| | backbone | nt 1-185 | | Biolabs |
| 185-222 | Linker | Not applicable | Synthetic | Not applicable |
| 223-800 | CMV | Not Applicable | pHCMV | (1994) PNAS |
| | | | | 91: 9564-68 |
| 801-1136 | R, U5, PBS, and | Accession #M19921.2 | pNL4-3 | Maldarelli, et. al. |
| | packaging sequences | nt 454-789 | | (1991) |
| | | | | J Virol: |
| | | | | 65(11): 5732-43 |
| 1137-1139 | Gag start codon (ATG) | Not Applicable | Synthetic | Not applicable |
| | changed to stop codon | | | |
| | (TAG) | | | |
| 1140-1240 | HIV-1 gag sequence | Accession #M19921.2 | pNL4-3 | Maldarelli, et. al. |
| | | nt 793-893 | | (1991) |
| | | | | J Virol: |
| | | | | 65(11): 5732-43 |
| 1241-1243 | HIV-1 gag sequence | Not Applicable | Synthetic | Not applicable |
| | changed to a second | | | |
| | stop codon | | | |
| 1244-1595 | HIV-1 gag sequence | Accession #M19921.2 | pNL4-3 | Maldarelli, et. al. |
| | | nt 897-1248 | | (1991) |
| | | | | J Virol: |
| | | | | 65(11): 5732-43 |
| 1596-1992 | HIV-1 pol | Accession #M19921.2 | pNL4-3 | Maldarelli, et. al. |
| | cPPT/CTS | nt 4745-5125 | | (1991) |
| | | | | J Virol: |
| | | | | 65(11): 5732-43 |
| 1993-2517 | HIV-1, isolate HXB3 | Accession #M14100.1 | PgTAT-CMV | Malim, M. H. |
| | env region (RRE) | nt 1875-2399 | | Nature (1988) |
| | | | | 335: 181-183 |
| 2518-2693 | HIV-1 env sequences | Accession #M19921.2 | pNL4-3 | Maldarelli, et. al. |
| | S/A | nt 8290-8470 | | (1991) |
| | | | | J Virol: |
| | | | | 65(11): 5732-43 |
| 2694-2708 | Linker | Not applicable | Synthetic | Not applicable |
| 2709-3096 | MND | Not applicable | pccl-c- | Challita et al. |
| | | | MNDU3c-x2 | (1995) |
| | | | | J. Virol. 69: |
| | | | | 748-755 |
| 3097-3124 | Linker | Not applicable | Synthetic | Not applicable |
| 3125-3187 | Signal peptide | Accession # | CD8a signal | Not applicable |
| | | NM_001768 | peptide | |
| 3188-3934 | BCMA02 scFv (V_{L}1- | Not applicable | Synthetic | Not applicable |
| | linker-V_{H}0) | | | |
| 3935-4141 | CD8a hinge and TM | Accession # | CD8a hinge | Milone et al |
| | | NM_001768 | and TM | (2009) |
| | | | | Mol Ther |
| | | | | 17(8): 1453-64 |
| 4144-4269 | CD137 (4-1BB) | Accession # | CD137 | Milone et al |
| | signaling domain | NM_001561 | signaling | (2009) |
| | | | domain | Mol Ther |
| | | | | 17(8): 1453-64 |
| 4270-4606 | CD3-ζ signaling | Accession # | CD3-ζ | Milone et al |
| | domain | NM_000734 | signaling | (2009) |
| | | | domain | Mol Ther |
| | | | | 17(8): 1453-64 |
| 4607-4717 | HIV-1 ppt and part of | Accession #M19921.2 | pNL4-3 | Maldarelli, et. al. |
| | 3′ U3 | nt 9005-9110 | | (1991) |
| | | | | J Virol: |
| | | | | 65(11): 5732-43 |
| 4718-4834 | HIV-1 part of U3 | Accession #M19921.2 | pNL4-3 | Maldarelli, et. al. |
| | (399 bp deletion) and R | nt 9511-9627 | | (1991) |
| | | | | J Virol: |
| | | | | 65(11): 5732-43 |
| 4835-4858 | Synthetic polyA | Not applicable | Synthetic | Levitt, N. Genes |
| | | | | & Dev (1989) |
| | | | | 3: 1019-1025 |
| 4859-4877 | Linker | Not applicable | Synthetic | Not Applicable |
| 4878-7350 | pUC19 backbone | Accession #L09137.2 | pUC19 | New England |
| | | nt 2636-2686 | | Biolabs |

### Example 2

### Anti-BCMA Car T Cell Manufacturing Process

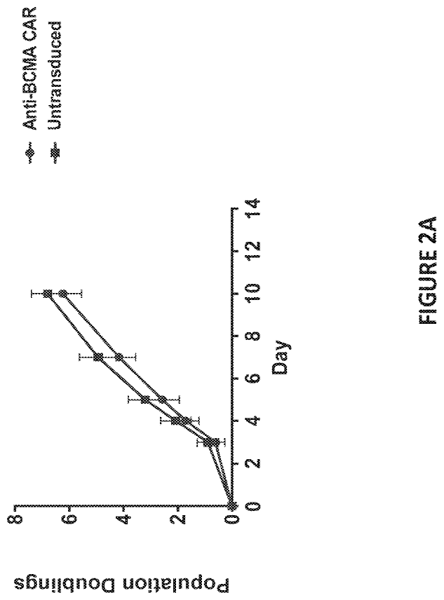

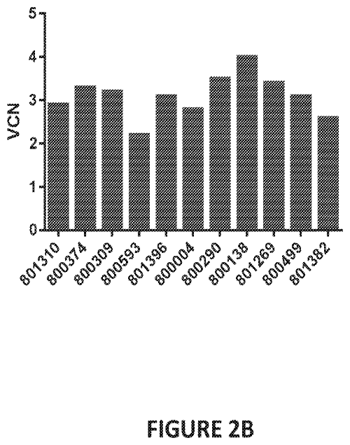

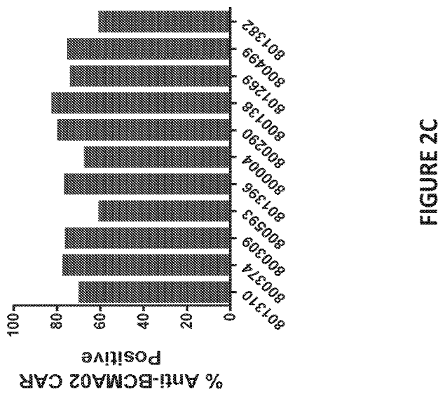

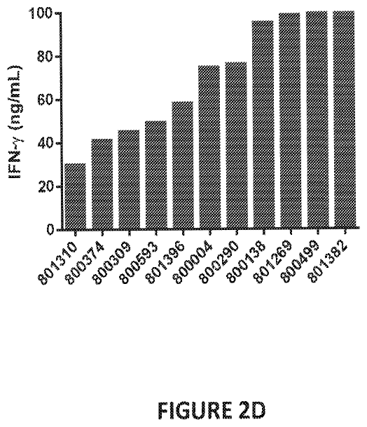

Unique anti-BCMA02 CAR T cell products are manufactured for each patient treatment. The reliability of the manufacturing process for anti-BCMA02 CAR T cell products was evaluated by generating anti-BCMA02 CAR T cells from 11 individual normal donor PBMC. Anti-BCMA02 CAR T cell expansion from each donor was comparable to a matched untransduced culture performed in parallel (FIG. 2A).

At the end of the culture period (day 10), T cell transduction efficiency was assessed by quantitating the number of integrated lentiviruses with qPCR and lentiviral-specific primer sets (vector copy number, VCN). Anti-BCMA02 CAR T cell cultures from the 11 donors showed comparable lentiviral transduction efficiency (FIG. 2B). The frequency of anti-BCMA02 CAR positive T cells was measured by flow cytometry and BCMA expression was found to be comparable across all donors (FIG. 2C).

The activity of each anti-BCMA02 CAR T cell product was assessed by IFNγ-release after co-culture with K562 cells engineered to express BCMA. All anti-BCMA CAR02 T cell products exhibited therapeutically relevant levels of IFNγ release when exposed to BCMA-expressing K562 cells (FIG. 2D).

### Example 3

### Anti-BCMA02 Car T Cells Show Therapeutic Activity in Human Clinical Trials of Relapsed/Refractory Multiple Myeloma

### Introduction

Chimeric antigen receptor (CAR) T cell therapies have demonstrated robust and sustained clinical responses observed in several hematologic malignancies. However, outside of CD19, clinical data supporting the promise of CAR T cells has been limited. The potential for CAR T cell safety and efficacy was tested in relapsed/refractory multiple myeloma (MM), a patient population with limited treatment options. To redirect T cells to MM we have targeted B cell maturation antigen (BCMA), a member of the tumor necrosis factor superfamily that is near uniformly expressed only by malignant myeloma cells, plasma cells, and some mature B cells. Initial proof of anti-BCMA activity has recently been demonstrated using T cells transduced with a gamma-retroviral vector encoding an anti-BCMA CAR with a CD28 costimulatory domain, but significant cytokine release syndrome occurred in patients with high disease burden (Ali et al., Blood 2016). Because the therapeutically effective dose of the anti-BCMA02 CAR T cells in humans is unpredictable, a dose-escalation trial was conducted.

### Methods

Anti-BCMA02 CAR T cells were administered to patients with relapsed and/or refractory BCMA-positive MM who received at least 3 prior regimens, including a proteasome inhibitor and an immunomodulatory agent or who were double-refractory. Briefly, peripheral blood mononuclear cells were collected via leukapheresis and transferred to a centralized manufacturing facility for transduction and expansion. Patients were lymphodepleted with cyclophosphamide 300 mg/m² and fludarabine 30 mg/m² on study Days −5, −4 and −3 and then received a single infusion of anti-BCMA02 CAR T cells on Day 0.

The average vector copy number (VCN) for the CAR T cells was determined for the nine patients. Table 4.

TABLE 4
CAR T cell VCN

| Patient | VCN |
|---|---|
| 1 | 2.08 |
| 2 | 1.97 |
| 3 | 2.11 |
| 4 | 5.98 |
| 5 | 3.01 |
| 6 | 4.13 |
| 7 | 3.38 |
| 8 | 4.01 |
| 9 | ND |

ND = not yet determined

### Results

Nine patients were infused with anti-BCMA02 CAR T cells in three dose cohorts of 5.0×10⁷ CAR+ T cells, 15.0×10⁷ CAR+ T cells, and 45.0×10⁷ CAR+ T cells. Cells were collected and successfully manufactured and released in all patients. Median age at enrollment for the 9 infused subjects was 58 years (43-68) and 67% were male. The median time from diagnosis was 6 years (1.3-8.6). The median number of prior lines of therapy was 6 (4-10), 100% of patients received at least 1 prior autologous stem cell transplant, 67% received prior daratumumab or CD38 mab, 89% received prior lenalidomide, 78% received prior pomalidomide, 100% received prior bortezomib, and 78% prior carfilzomib.

To date no dose limiting toxicities, neurotoxicities>Grade 2, or Grade 3 or higher cytokine release syndrome (CRS) have been observed. Moreover, no patients have required treatment with vasopressors, tocililuzmab or corticosteroids. Grade 1 or 2 CRS was reported in 6/9 (67%) treated patients. In addition to CRS, the most common treatment emergent adverse events were neutropenia (89%), leukopenia (67%) and anemia (44%).

Clinical responses were observed in every dose cohort. After the initial cohort, all patients remained on study and the overall response rate in the 6 patients treated at 15.0×10⁷ CAR+ T cells or higher was 83%, including 2 stringent complete responses (CRs) (see Table 5). Surprisingly, at dose levels above 5.0×10⁷ CAR+ T cells, all patients with bone marrow involvement at baseline have had no bone marrow disease detected at any point after day 14.

TABLE 5
Response by Dose Level & Duration

| | | | Time since |
|---|---|---|---|
| | | | bb2121 |
| | | | infusion |
| Dose | Patient | Response | (weeks) |
| 5.0 × 10⁷ | 1 | PR | 13 |
| | 2* | SD | 9 |
| | 3* | PD | 8 |
| 15.0 × 10⁷ | 4 | sCR | 28+ |
| | 5 | sCR | 21+ |
| | 6* | VGPR | 19+ |
| 45.0 × 10⁷ | 7 | PR | 13+ |
| | 8 | SD | 11+ |
| | 9* | PR | 4+ |

*indicates patient had ≥50% bone marrow involvement at baseline.
Responses graded using IMWG Uniform Response Criteria for Multiple Myeloma.
PR: partial response, SD: stable disease, PD: progressive disease, sCR: stringent complete response, VGPR: very good partial response, NE: not yet evaluated.
+denotes ongoing response.

CAR+ T cell expansion was consistently demonstrated, and is similar to other published CAR T cell trials. In the 5 patients treated with >5.0×10⁷ CAR+ T cells for whom data was available, the range of peak CAR+ T cells by flow cytometry was 10 to 686 CAR+ cells/L in peripheral blood and 4 to 527 CAR+ cells/L in bone marrow, and the range of peak copies/g genomic DNA in peripheral blood was 34,231 to 860,204 by PCR.

### Conclusions

Anti-BCMA02 CAR T cells showed remarkable efficacy at dose levels above 5.0×10⁷ CAR+ T cells, including 2 CRs and ongoing clinical response at 6 months. In contrast to results with other CAR T cell therapies, the efficacy of the anti-BCMA02 CAR T cells was accompanied by unexpectedly mild and manageable CRS, including in patients with >50% bone marrow involvement. These data support the therapeutic efficacy of anti-BCMA02 CAR T cells for relapsed/refractory multiple myeloma.

Additional dose cohorts are planned with dose levels of 80.0×10⁷ and 12.0×10⁸ CAR+ T cells.

### Example 4

### Anti-BCMA02 Car T Cells Continue to Show Therapeutic Activity in Human Clinical Trials of Relapsed/Refractory Multiple Myeloma

Twenty one patients have been infused with anti-BCMA02 CAR T cells in three dose cohorts of 5.0×10⁷ CAR+ T cells (3 patients), 15.0×10⁷ CAR+ T cells (6 patients), 45.0×10⁷ CAR+ T cells (9 patients), and 80.0×10⁷ CAR+ T cells (3 patients). Cells were collected and successfully manufactured and released in all patients. Median age at enrollment for the 9 infused subjects was 58 years (37-74) and 62% were male. The median time from diagnosis was 5 years (1.0-16.0). The median number of prior lines of therapy was 7 (3-14), 100% of patients received at least 1 prior autologous stem cell transplant, 100% previously treated with lenalidomide and bortezomib, 91% previously treated with pomalidomide and carfilzomib, 71% previously treated with daratumumab, and 29% of patients were penta-refractory (bortezomib, lenalidomide, carfilzomib, pomalidomide, daratumumab).

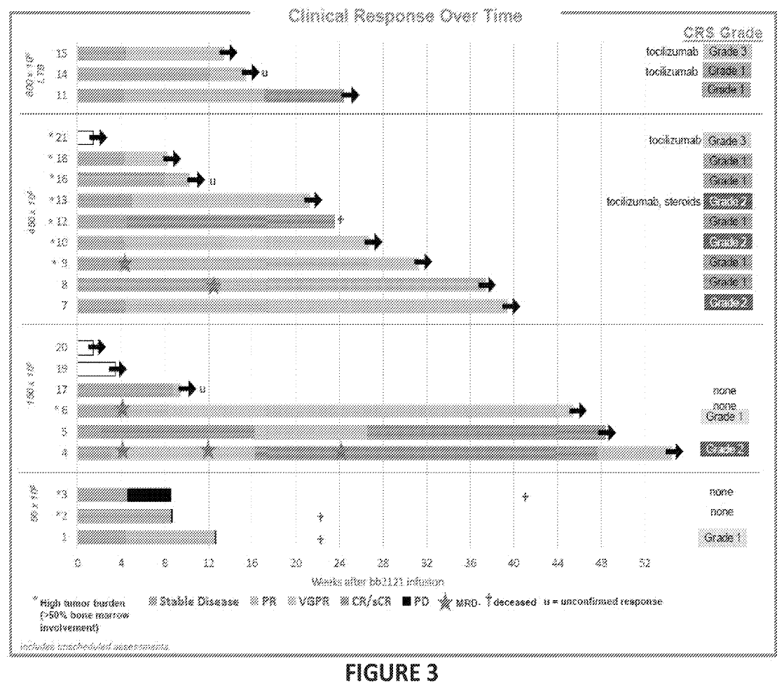

All patients in active dose cohorts achieved an objective response, duration up to 54 weeks. FIG. 3. CAR T cell dose, number of evaluable patients, overall response rate, best response, median lines of prior therapy, and safety for each cohort are presented in Table 6.

TABLE 6
Cohort Data

| Cohort | 1 | 2 | 3 | 4 |
|---|---|---|---|---|
| CAR+ T Cell | 50 × 10⁶ | 150 × 10⁶ | 450 × 10⁶ | 800 × 10⁶ |
| Dose | | | | |
| Number of | 3 | 4 | 8 | 3 |
| Patients | | | | |
| Evaluable for | | | | |
| Efficacy | | | | |
| Overall | 33% | 100% | 100% | 100% |
| Response Rate | | | | |
| in Cohort | | | | |
| Best Response | PD (1 patient) | CR (2 patients, | CR (1 patient*) | VGPR (1 patient) |
| | SD (1 patient) | 1 patient | VGPR (5 patients; | PR (1 patient) |
| | PR (1 patient) | MRD negative) | 1 patient | CR (1 patient) |
| | | VGPR (1 patient | MRD negative) | |
| | | MRD negative) | PR (2 patients; | |
| | | PR (1 patient) | 1 patient | |
| | | | MRD negative) | |
| | | | *Patient died of | |
| | | | unrelated cardio | |
| | | | pulmonary arrest | |

| | |
|---|---|
| Bone Marrow | All patients in cohorts 2, 3 and 4 with bone marrow |
| Involvement | involvement at baseline had no detectable multiple |
| | myeloma cells in their bone marrow on Day 14 or |
| | beyond. Of four patients evaluable for MRD status, |
| | all four were found to be MRD-negative. |

| | |
|---|---|
| Median Prior | 7 (range: 3-14); all patients had at least one prior autologous |
| Lines of | stem cell transplant, as well as prior exposure to a proteasome |
| Therapy | inhibitor and an immunomodulatory agent; 71% of patients had |
| | previously received daratumumab or CD38 antibody |
| Safety | 15/21 (71%) of patients had CRS, mostly Grade 1 & 2; 2 patients |
| | with Grade 3 CRS that resolved within 24 hours. 4 patients received |
| | tocilizumab, 1 (Grade 2 CRS) received steroids. The most common |
| | treatment-emergent Grade 3-4 AEs in 21 infused patients include |
| | cytopenias commonly associated with cy/flu lymphodepletion, as well |
| | as Grade 3 events of hyponatraemia (n = 4), cytokine release |
| | syndrome (n = 2), upper respiratory infection (n = 2), and syncope (n = 2). |

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A composition comprising a pharmaceutically acceptable carrier and a therapeutically effective amount of anti-B cell maturation antigen (BCMA) chimeric antigen receptor (CAR) T cells, wherein the therapeutically effective amount is greater than 45.0×10⁷ to less than 80×10⁷ anti-BCMA CAR T cells, wherein the anti-BCMA CAR comprises the amino acids 22-493 of the amino acid sequence set forth in SEQ ID NO: 9, and wherein the composition is formulated for intravenous administration in a single dose.

2. The composition of claim 1, comprising 50:50 PlasmaLyte A to CryoStor CS10.

3.-10. (canceled)

11. The composition of claim 1, wherein the anti-BCMA CAR comprises the amino acid sequence of SEQ ID NO: 9.

12. The composition of claim 1, wherein the anti-BCMA CAR is encoded by the nucleotide sequence set forth in SEQ ID NO: 10.

13. The composition of claim 1, wherein the anti-BCMA CAR T cells were produced by transduction with a lentiviral vector encoding the anti-BCMA CAR.

14. The composition of claim 13, wherein the lentiviral vector is a human immunodeficiency virus vector.

15. The composition of claim 1, wherein the T cells comprise CD8+ T cells.

16. A composition comprising a pharmaceutically acceptable carrier and a therapeutically effective amount of anti-B cell maturation antigen (BCMA) chimeric antigen receptor (CAR) T cells, wherein the therapeutically effective amount is 45.0×10⁷±20% anti-human BCMA CAR T cells, wherein the anti-BCMA CAR comprises amino acids 22-493 of the amino acid sequence set forth in SEQ ID NO: 9, and wherein the composition is formulated for intravenous administration in a single dose.

17. The composition of claim 16, comprising 50:50 PlasmaLyte A to CryoStor CS10.

18. The composition of claim 16, wherein the anti-BCMA CAR comprises the amino acid sequence of SEQ ID NO: 9.

19. The composition of claim 16, wherein the anti-BCMA CAR is encoded by the nucleotide sequence set forth in SEQ ID NO: 10.

20. The composition of claim 16, wherein the anti-BCMA CAR T cells were produced by transduction with a lentiviral vector encoding the anti-BCMA CAR.

21. The composition of claim 20, wherein the lentiviral vector is a human immunodeficiency virus vector.

22. The composition of claim 16, wherein the T cells comprise CD8+ T cells.


SEQUENCE LISTING (36 sequences)

SEQ ID NO: 1 - protein, 15 aa, Mus musculus
RASESVTILGSHLIH

SEQ ID NO: 2 - protein, 7 aa, Mus musculus
LASNVQT

SEQ ID NO: 3 - protein, 9 aa, Mus musculus
LQSRTIPRT

SEQ ID NO: 4 - protein, 5 aa, Mus musculus
DYSIN

SEQ ID NO: 5 - protein, 17 aa, Mus musculus
WINTETREPAYAYDFRG

SEQ ID NO: 6 - protein, 8 aa, Mus musculus
DYSYAMDY

SEQ ID NO: 7 - protein, 111 aa, Mus musculus
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 8 - protein, 117 aa, Mus musculus
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 9 - protein, 493 aa, synthetic construct
  REGION 1..493: note = anti-BCMA02 CAR
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1485 bp, synthetic construct
  misc_feature 1..1485: note = anti-BCMA02 CAR
  source 1..1485: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 11 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 12 - protein, 5 aa, synthetic construct
  REGION 1..5: note = Flexible peptide linker
DGGGS

SEQ ID NO: 13 - protein, 5 aa, synthetic construct
  REGION 1..5: note = Flexible peptide linker
TGEKP

SEQ ID NO: 14 - protein, 4 aa, synthetic construct
  REGION 1..4: note = Flexible peptide linker
GGRR

SEQ ID NO: 15 - protein, 5 aa, synthetic construct
  REGION 1..5: note = Flexible peptide linker
GGGGS

SEQ ID NO: 16 - protein, 14 aa, synthetic construct
  REGION 1..14: note = Flexible peptide linker
EGKSSGSGSESKVD

SEQ ID NO: 17 - protein, 18 aa, synthetic construct
  REGION 1..18: note = Flexible peptide linker
KESGSVSSEQLAQFRSLD

SEQ ID NO: 18 - protein, 8 aa, synthetic construct
  REGION 1..8: note = Flexible peptide linker
GGRRGGGS

SEQ ID NO: 19 - protein, 9 aa, synthetic construct
  REGION 1..9: note = Flexible peptide linker
LRQRDGERP

SEQ ID NO: 20 - protein, 12 aa, synthetic construct
  REGION 1..12: note = Flexible peptide linker
LRQKDGGGSERP

SEQ ID NO: 21 - protein, 16 aa, synthetic construct
  REGION 1..16: note = Flexible peptide linker
LRQKDGGGSGGGSERP

SEQ ID NO: 22 - protein, 18 aa, synthetic construct
  REGION 1..18: note = Flexible peptide linker
GSTSGSGKPGSGEGSTKG

SEQ ID NO: 23 - skipped

SEQ ID NO: 24 - protein, 7 aa, synthetic construct
  REGION 1..7: note = protease cleavage site
ENLYFQG

SEQ ID NO: 25 - protein, 7 aa, synthetic construct
  REGION 1..7: note = protease cleavage site
ENLYFQS

SEQ ID NO: 26 - protein, 19 aa, synthetic construct
  REGION 1..19: note = protease cleavage site
LLNFDLLKLAGDVESNPGP

SEQ ID NO: 27 - protein, 19 aa, synthetic construct
  REGION 1..19: note = protease cleavage site
TLNFDLLKLAGDVESNPGP

SEQ ID NO: 28 - protein, 14 aa, synthetic construct
  REGION 1..14: note = protease cleavage site
LLKLAGDVESNPGP

SEQ ID NO: 29 - protein, 17 aa, synthetic construct
  REGION 1..17: note = protease cleavage site
NFDLLKLAGDVESNPGP

SEQ ID NO: 30 - protein, 20 aa, synthetic construct
  REGION 1..20: note = protease cleavage site
QLLNFDLLKLAGDVESNPGP

SEQ ID NO: 31 - protein, 24 aa, synthetic construct
  REGION 1..24: note = protease cleavage site
APVKQTLNFDLLKLAGDVESNPGP

SEQ ID NO: 32 - protein, 40 aa, synthetic construct
  REGION 1..40: note = protease cleavage site
VTELLYRMKRAETYCPRPLLAIHPTEARHKQKIVAPVKQT

SEQ ID NO: 33 - protein, 18 aa, synthetic construct
  REGION 1..18: note = protease cleavage site
LNFDLLKLAGDVESNPGP

SEQ ID NO: 34 - protein, 40 aa, synthetic construct
  REGION 1..40: note = protease cleavage site
LLAIHPTEARHKQKIVAPVKQTLNFDLLKLAGDVESNPGP

SEQ ID NO: 35 - protein, 33 aa, synthetic construct
  REGION 1..33: note = protease cleavage site
EARHKQKIVAPVKQTLNFDLLKLAGDVESNPGP

SEQ ID NO: 36 - DNA, 7350 bp, synthetic construct
  misc_feature 1..7350: note = anti-BCMA02 CAR vector
  source 1..7350: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatcatatgccagcctatggtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaattaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatagctctagagcgatcccgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccatctcgacggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgattcacctgacgcgtctacgccaccatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatgacaggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaatgtgtgtgttggttttttgtgtgtcgaaattctagcgattctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggactagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgagcttgcatgccgacattgattattgactagtccctaagaaaccattcttatcatgacattaacctataaaaataggcgtatcacgaggccctttcgtc